(19) United States
(12) Patent Application Publication
(10) Pub. No.: US 2012/0165558 A1
(43) Pub. Date: Jun. 28, 2012

(54) REGULARLY STACKED MULTILAMELLAR AND RANDOMLY ALIGNED UNILAMELLAR ZEOLITE NANOSHEETS, AND THEIR ANALOGUE MATERIALS WHOSE FRAMEWORK THICKNESS WERE CORRESPONDING TO ONE UNIT CELL SIZE OR LESS THAN 10 UNIT CELL SIZE

(75) Inventors: Ryong Ryoo, Daejeon (KR); Minkee Choi, Daejeon (KR); Kyungku Na, Daejeon (KR)

(73) Assignee: KOREA ADVANCED INSTITUTE OF SCIENCE AND TECHNOLOGY, Daejeon (KR)

(21) Appl. No.: 13/380,505

(22) PCT Filed: Jun. 11, 2010
(86) PCT No.: PCT/KR2010/003759

§ 371 Date: Mar. 19, 2012

(30) Foreign Application Priority Data
Jun. 22, 2009 (KR) 10-2009-0055534

(51) Int. Cl. C07D 311/32; C01B 39/02; C07C 1/20; C01B 39/54; C01B 39/00; B01J 29/04

(52) U.S. Cl. 549/403; 423/700; 423/702; 502/60; 585/408; 585/639

(57) ABSTRACT

The present invention relates to microporous molecular sieve materials and their analogue molecular sieve materials having a crystalline unilamellar or multilamellar framework with a single unit cell thickness in which layers are aligned regularly or randomly, the molecular sieve materials being synthesized by adding an organic surfactant to the synthesis composition of zeolite. In addition, the present invention relates to micro-mesoporous molecular sieve materials activated or functionalized by dealumination, ion exchange or other post treatments, and the use thereof as catalyst. These novel materials have dramatically increased external surface area by virtue of their framework with nano-scale thickness, and thus exhibit improved molecular diffusion, and thus have much higher activities as catalyst and ion exchange resin than conventional zeolites. In particular, the materials of the present invention exhibit high reactivity and dramatically increased catalyst life in various organic reactions such as carbon-carbon coupling, alkylation, acylation, etc. of organic molecules.

### TECHNICAL FIELD

The present invention relates to MFI (3-letter code by the International Zeolite Association) zeolites and their analogue molecular sieve materials having a unilamellar or multilamellar structure with the framework thickness of a single unit cell, and a method for preparing the materials. Specifically, the present invention relates to materials having a framework with a single unit cell thickness comprising a randomly aligned unilamellar structure, materials having a framework with a single unit cell thickness comprising regularly aligned multilamellar stacking, and a method for preparing the materials. The materials of the present invention include not only materials whose framework comprises one single unit cell, but also materials whose framework is formed by a connection of 10 or less single unit cells. In addition, the present invention relates to novel zeolite materials prepared by adding an organic surfactant having 2 or more amine or ammonium functional groups to the synthesis composition of zeolite, a method for preparing the materials, and application of thus obtained zeolites and their analogue molecular sieve materials as catalyst.

### BACKGROUND ART

A zeolite is defined as a crystalline aluminosilicate material with a framework structure comprising regularly aligned micropores of a molecular size (0.3<diameter<2 nm). Because zeolite has micropores with a diameter in the dimension of the size of molecules, zeolite can serve as a molecular sieve capable of selectively adsorbing and diffusing molecules. By virtue of such molecular sieve effects, zeolite allows for molecular specific adsorption, ion exchange and catalytic reactions (C. S. Cundy, et al., Chem. Rev. 2003, 103, 663). However, since the diameter of zeolite micropores is very small, the molecular diffusion rate in zeolite is low, which restricts a reaction rate in many applications. Accordingly, there have been attempts to facilitate the molecular diffusion into zeolite micropores by increasing the external surface area of zeolite particles by reducing the thickness of a zeolite framework.

In order to synthesize zeolite having a large specific surface area, there have been attempts to synthesize zeolite in small crystal size with a nanometer thickness. A method of synthesizing zeolite in colloidal form with a nanometer size (10 nm or more) by adjusting the synthesis composition of the zeolite and lowering crystallization temperature (L. Tosheva et al., Chem. Mater. 2005, 17, 2494). However, such synthesis methods, the obtained zeolites have low crystallinity, the yield is low, and there is a limitation that thus synthesized zeolites have to be separated by centrifugation, not filtering. There has been another attempt to increase the specific surface area of zeolite by creating pores with larger diameter, i.e., mesopores (2<diameter<50 nm) and macropores (50 nm<diameter), in zeolite crystals. Anderson and his co-researchers synthesized zeolite with macropores by crystallizing diatomite using the zeolite seed crystal (Anderson, M. W. et al., Angew. Chem. Int. Ed. 2000, 39, 2707). Recently, a method of creating mesopores in zeolite crystals by synthesizing zeolite in various solid templates, such as carbon nanoparticles, nano fibers and spherical polymers, and calcining the template has been published. Stein and his co-researchers have published technology of synthesizing a mesoporous molecular sieve by using spherical polystyrene with uniform size of about 100μ (U.S. Pat. No. 6,680,013 B1). Jacobson has synthesized mesoporous zeolite with wide pore size distribution of 10-100 nm (U.S. Pat. No. 6,620,402 B2) using carbon as template. In addition, it has been reported that such materials prepared using a solid matrix have improved catalytic activities because mesopores allow for better molecular diffusion (Christensen, C. H. et al., J. Am. Chem. Soc. 2003, 125, 13370). Recently, a technology of creating mesopores in zeolite crystals by adding organosilane to the synthesis composition of zeolite has been published (Korean Patent No. 10-0727288). In addition, Corma and his researchers have published a method of delaminating FER zeolite and MWW zeolite, having a lamellar structure, into a unilamellar thin layer (A. Corma et al., Nature 1998, 396, 353; A. Corma et al., Spanish Patent No. 9502188 (1996), PCT-WO Patent 97/17290 (1997)).

### DISCLOSURE OF INVENTION

### Technical Problem

As described above, the intramolecular diffusion into zeolite can be maximized by synthesizing a zeolite having a framework as thin as the thickness of 10 or less single unit cells and possessing dramatically increased specific surface area. In principle, zeolites will exhibit maximized molecular diffusion if the thickness of the zeolite crystal is reduced to the single unit cell dimension. However, thermodynamically, the actual synthesis of a zeolite material with a single unit cell thickness is extremely difficult. Zeolite crystallization involves a process that minimizes the surface energy of crystals, resulting in growing crystals to a size larger than a certain size (Ostwald ripening). This phenomenon becomes more significant as the crystal size decreases. Due to this phenomenon, although conventional synthesis methods can synthesize a zeolite with the framework thickness of about 5-100 nm, the conventional synthesis methods cannot synthesize a zeolite with a single unit cell thickness or a ultrathin zeolite having a framework with nano-size thickness comprising 10 or less single unit cells. Thus, the present inventors have conducted studies to prepare zeolite materials or their analogue molecular sieve materials having an ultrathin lamellar stacking which has the thickness of a single unit cell or that of 10 or less single unit cells. As a result, the present inventors have confirmed that a zeolite having a nanosized framework with a single unit cell thickness can be synthesized by adding a structure-directing organic surfactant having 2 or more ammonium functional groups to a zeolite synthesis solution, and completed the present invention. As such, the objective of the present invention is to provide zeolites having a framework with a single unit cell thickness and a method for preparing the same. In addition, the present invention relates to the application of thus obtained materials as catalyst. In addition, the present invention relates to zeolites having a lamellar structure with the thickness of the stacking of a plurality of single unit cells, prepared by adjusting the number of ammonium or amine functional groups of organic surfactant, and a method for preparing the same. In addition, according to the present invention, it is possible to synthesize not only MFI zeolite but also MTW zeolite by adjusting the structure of organic surfactant, and further, it is possible to synthesize even aluminophosphate (AlPO), which is a zeotype material. In addition, according to the synthesis method of the present invention, it is possible to synthesize other zeolite or zeotype materials than MFI zeolite, MTW zeolite and AlPO.

### Solution to Problem

The present inventors added an organic surfactant having a plurality of ammonium functional groups to a zeolite synthesis gel, crystallized the mixture under acidic or basic condition, and then selectively removed organic materials to obtain various zeolite materials and their analogue materials having a unilamellar or multilamellar structure which has a single unit cell thickness or comprises the stacking of 10 or less single unit cells. Here, “analogue material” refer to a material obtained by subjecting the novel zeolite material according to the present invention to a common post-treatment method such as pillaring, delamination, dealumination, alkali treatment, cation exchange, etc., and the “analogue material” is different from the above-described zeotype material. Hereinafter, we will explain in more detail each step of the method for preparing novel zeolite materials and their analogue materials.

Step 1: An organic-inorganic hybrid gel is synthesized by polymerizing an organo-functionalized silica precursor with another gel precursor such as silica or alumina. In this case, hydrophobic organic domains are self-assembled and are formed between inorganic domains by non-covalent force such as van der Waals force, dipole-dipole interaction, ionic interaction, etc. Gel domains are continuously or locally aligned in regular manner depending on the type and concentration of organic materials.

Step 2: Inorganic gel domains with nano size stabilized by organic domains are converted to a unilamellar or multilamellar zeolite which has a single unit cell thickness or comprises the stacking of 10 or less single unit cells, by a crystallization process depending on the type of organic surfactant and the number of ammonium functional groups included in the organic surfactant. In this case, due to the effect of stabilization by the organic materials surrounding each zeolite, further growth of zeolite is suppressed and the size of zeolite crystals is controlled to be 10 nm or less. The crystallization process can be carried out by any conventional method including hydrothermal synthesis, dry-gel synthesis, microwave synthesis, etc.

Step 3: After the crystallization process, zeolite can be obtained by a common method such as filtering, centrifugation, etc. Thus obtained material is subjected to calcination or a chemical reaction to selectively remove organic materials in total or in part. The pure organic surfactant used in the present invention, having two ammonium functional groups, or both an ammonium functional group and an amine functional group, can be expressed as the following formula [1] or [2]:

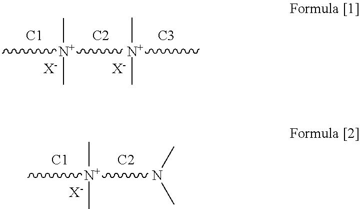

(wherein X is halogen (Cl, Br, I) or hydroxide group (OH), each of C1, C2 and C3 is independently substituted or unsubstituted alkyl group or C3 is alkenyl group or may be various molecular structures substituted with other atom except carbon in periodic table. Ammonium functional group may be extended to 2 or more and may be extended to material with more various structure and C1 comprises 8˜22 carbon atoms, C2 comprises 3˜6 carbon atoms and C3 comprises 1˜8 carbon atoms.)

In the present invention, an organic surfactant is expressed in a general form as: the number of carbon atoms of C1—the number of carbon atoms of C2—the number of carbon atoms of C3 (ex. 22-6-6: organic surfactant having 22 carbon atoms in C1, 6 carbon atoms in C2, 6 carbon atoms in C3, and 2 ammonium functional groups; 22-6-0: organic surfactant having 22 carbon atoms in C1, 6 carbon atoms in C2, one ammonium functional group and one amine functional group). In case where the substituent X is hydroxide, not halogen, the expression “(OH—)” follows the general expression. In particular, the present invention has found for the first time that the number of single unit cells included in one unilamellar structure can be controlled by adjusting the structure of organic surfactant or the number of ammonium or amine functional groups therein. The most important factor in the synthesis of the unilamellar or multilamellar zeolite which has a single unit cell thickness or comprises the stacking of 10 or less single unit cells according to the present invention is that an organic surfactant capable of self-assembly in the formation of organic-inorganic hybrid gel and having 2 or more ammonium functional groups is used. When such organic surfactant is added to a zeolite synthesis gel, two ammonium functional groups introduce the formation of a zeolite framework, and hydrophobic alkyl tails suppress further growth of the zeolite. In addition, the hydrophobic alkyl tails contribute to the self-assembly of the obtained lamellar zeolite structure and thus the formation of mesopores (2<diameter<50 nm) between zeolite crystals.

The materials synthesized according to the present invention exhibit characteristic X-ray diffraction and electron diffraction patterns corresponding to the microporous structures of zeolite. In addition, the present inventors confirmed that the materials of the present invention include not only micropores intrinsic to zeolite but also mesopores with high pore volume by using a nitrogen adsorption method. In addition, the present inventors find that the crystalline framework comprising micropores is a randomly aligned unilamellar structure or regularly aligned multilamellar stacking which has a single unit cell thickness or which comprises stacking of 10 or less single unit cells, by using a transmission electron microscope (TEM). Thus, it was confirmed that in the materials of the present invention, micropores are regularly arranged, and mesopores are randomly or regularly arranged. The zeolites synthesized according to the present invention have a very large specific surface area (500˜800 m²/g) due to their nanosized framework, which is dramatically higher than the specific surface area of conventional MFI zeolite (300˜450 m²/g). The present inventors also confirmed that the materials of the present invention are in a perfect crystalline phase, and that an amorphous phase has not been created separately, by using a scanning electron microscope. The zeolites prepared according to the present invention show ²⁷Al MAS NMR peaks in the range of 50˜60 ppm due to Al included in the framework of the zeolites, but no peak was observed in the range of 0˜10 ppm corresponding to the peaks of Al located outside of a zeolite framework. The X-ray diffraction and NMR data indicate that the novel materials of the present invention have a perfect crystalline structure having uniform chemical environment around Al sites.

### Advantageous Effects of Invention

As explained above and confirmed below, the present invention provides a method for preparing zeolites and their analogue molecular sieve materials having a multilamellar or unilamellar structure with a single unit cell thickness. As evidenced in the present application, the materials of the present invention are a MFI zeolite material having a multilamellar or unilamellar structure with a single unit cell thickness, a MTW zeolite material and aluminophosphate (AIPO) material having a multilamellar or unilamellar structure with a nano-size thickness of 10.0 nm or less. The zeolite materials and zeotype materials of the present invention have remarkably increased surface area as compared with conventional zeolite materials, and thus exhibit significantly increased molecular diffusion rate and significantly improved catalytic activities. In addition, the materials of the present invention exhibit very high activities in the adsorption, separation and catalytic reaction of macro organic molecules and the reforming of petroleum. By virtue of the different framework thickness from those of conventional zeolite materials, the materials of the present invention are expected to be applied in various industrial and scientific fields and exhibit new properties.

### BRIEF DESCRIPTION OF DRAWINGS

FIG. 1 shows SEM images of the multilamellar MFI aluminosilicate with a single unit cell thickness prepared according to Example 1 before calcination.

FIG. 2 shows TEM images of the multilamellar MFI aluminosilicate with a single unit cell thickness prepared according to Example 1 before calcination.

FIG. 3 shows a TEM image (see (a)) and electron diffraction pattern (see (b)) of the wide plane of the multilamellar MFI aluminosilicate with a single unit cell thickness prepared according to Example 1 before calcination.

FIG. 4 shows low-angle X-ray diffraction data of the multilamellar MFI aluminosilicate with a single unit cell thickness prepared according to Example 1 before calcination.

FIG. 5 shows high-angle X-ray diffraction data of the multilamellar MFI aluminosilicate with a single unit cell thickness prepared according to Example 1 before calcination.

FIG. 6 shows the ²⁷Al MAS NMR spectrum of the multilamellar MFI aluminosilicate with a single unit cell thickness prepared according to Example 1 before calcination.

FIG. 7 shows TEM images of the multilamellar MFI aluminosilicate with a single unit cell thickness prepared according to Example 2 after calcination.

FIG. 8 shows the nitrogen adsorption isotherm of the multilamellar MFI aluminosilicate with a single unit cell thickness prepared according to Example 2 after calcination.

FIG. 9 shows a TEM image of the multilamellar MFI aluminosilicate with a single unit cell thickness supported by silica pillars prepared according to Example 3 after calcination.

FIG. 10 shows a TEM image of the delaminated unilamellar MFI aluminosilicate with a single unit cell thickness according to Example 4 after calcination.

FIG. 11 shows low-angle X-ray diffraction data of the multilamellar MFI aluminosilicate with a single unit cell thickness prepared according to Example 5 before calcination.

FIG. 12 shows high-angle X-ray diffraction data of the multilamellar MFI aluminosilicate with a single unit cell thickness prepared according to Example 5 after calcination.

FIG. 13 shows high-angle X-ray diffraction data of the multilamellar MFI silicate with a single unit cell thickness prepared according to Example 6 after calcination.

FIG. 14 shows high-angle X-ray diffraction data of the multilamellar MFI titanosilicate with a single unit cell thickness prepared according to Example 7 after calcination.

FIG. 15 shows SEM images of the unilamellar MFI aluminosilicate with a single unit cell thickness prepared according to Example 8 after calcination.

FIG. 16 shows TEM images of the unilamellar MFI aluminosilicate with a single unit cell thickness prepared according to Example 8 after calcination.

FIG. 17 shows the nitrogen adsorption isotherm of the unilamellar MFI aluminosilicate with a single unit cell thickness prepared according to Example 8 after calcination.

FIG. 18 shows a SEM image of the multilamellar MTW aluminosilicate with a framework thickness of 5.0 nm or less prepared according to Example 9 after calcination.

FIG. 19 shows a TEM image of the multilamellar MTW aluminosilicate with a framework thickness of 5.0 nm or less prepared according to Example 9 after calcination.

FIG. 20 shows high-angle X-ray diffraction data of the multilamellar MTW aluminosilicate with a framework thickness of 5.0 nm or less prepared according to Example 9 after calcination.

FIG. 21 shows high- and low-angle X-ray diffraction data of the multilamellar aluminophosphate with a framework thickness of 5.0 nm or less prepared according to Example 10 before calcination.

FIG. 22 shows a TEM image of the multilamellar aluminophosphate with a framework thickness of 5.0 nm or less prepared according to Example 10 before calcination.

### BEST MODE FOR CARRYING OUT THE INVENTION

Hereinafter, the present invention will be described in further detail with reference to Examples. However, it should be understood that the Examples are for the purpose of illustration and to describe embodiments of the best mode of the invention at the present time. The scope of the invention is not in any way limited by the examples set forth below.

### Example 1

### Synthesis of Multilamellar MFI Aluminosilicate with a Single Unit Cell Thickness

Organic surfactant 22-6-6 (organic surfactant of formula [1] having 22 carbon atoms in C1, 6 carbon atoms in C2, 6 carbon atoms in C3, and 2 ammonium functional groups) were mixed with tetraethylorthosilicate (TEOS), NaOH, Al₂(SO₄)₃, H₂SO₄ and distilled water to prepare a gel mixture with the following molar composition:

1 Al₂O₃: 30 Na₂O: 100 SiO₂: 4000 H₂O: 18 H₂SO₄: 10 22-6-6 organic surfactant

After stirring the mixed gel at room temperature for three hours, the final mixed product was placed in a stainless autoclave, and then was left at 150° C. for five days. After cooling the autoclave to room temperature, the product was filtered and washed for several times. The product obtained was dried at 110° C.

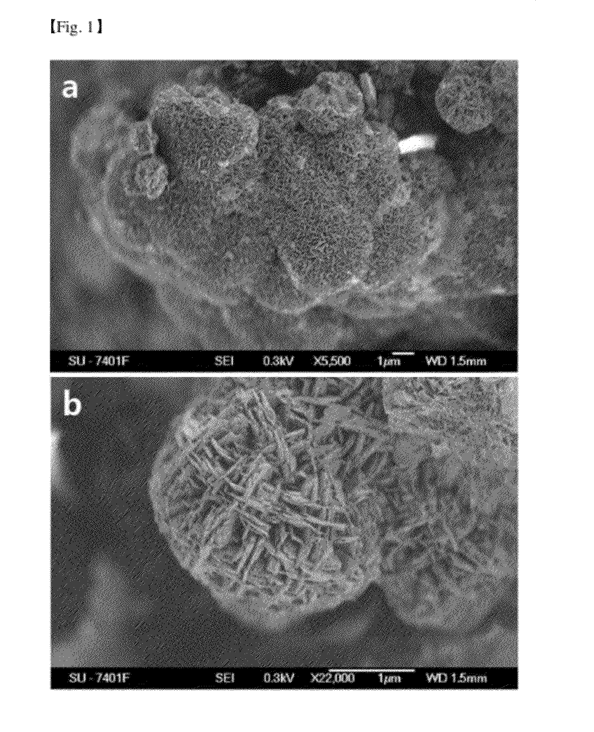

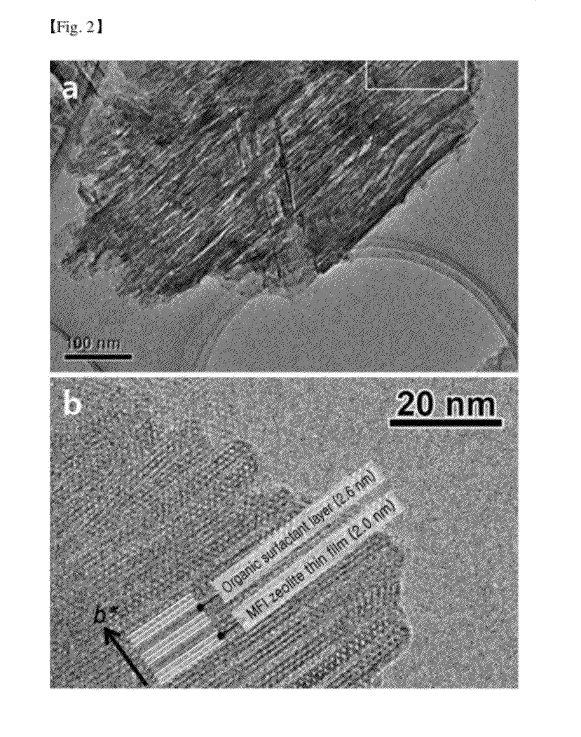

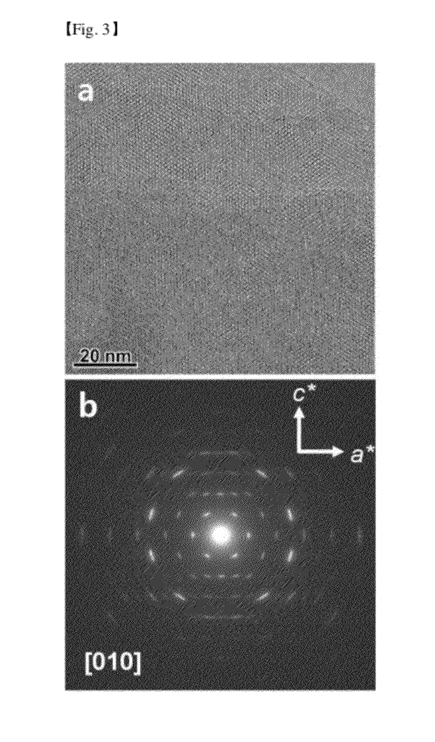

The SEM image of zeolite synthesized as above shows that the zeolite has grown as a crystal having a shape of lamellar structure with a thickness with nano unit (20-50 nm) (FIG. 1). FIG. 2 is a TEM image of the cross-section of such lamellar-structure shaped crystal showing that each lamellar-shaped crystal is stacked up in a zeolite thin film of 2.0 nm and a surfactant layer of 2.6 nm, alternately to form multilamellar stacking. Also, FIG. 2 shows that the zeolite thin film and surfactant layer are stacked perpendicularly to the b-axis of the MFI crystal structure. FIG. 3 is a TEM image of the wide section of the lamellar structure crystal, and electron diffraction pattern, showing that the wide section of the zeolite is a-c plane of the zeolite crystal section, i.e. (010) plane. According to the electron microscope analysis, the present material is formed in a multilamellar stacking wherein the zeolite thin films whose a-c plane is wide and the thickness toward b-axis corresponds to a single unit cell thickness (2.0 nm) are aligned regularly.

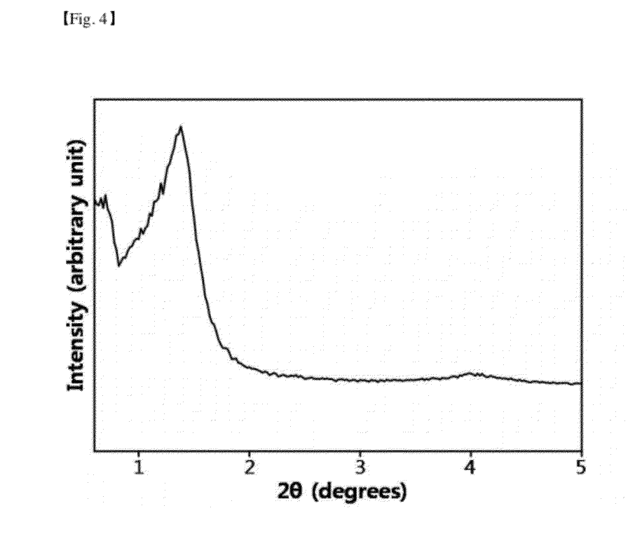

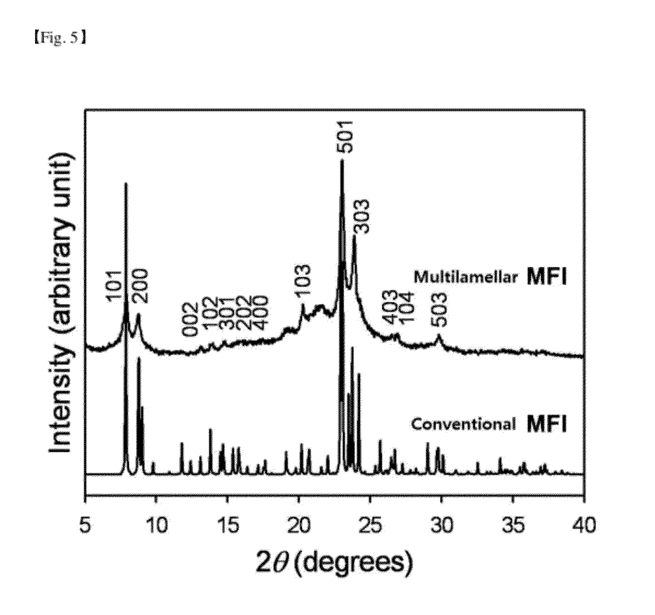

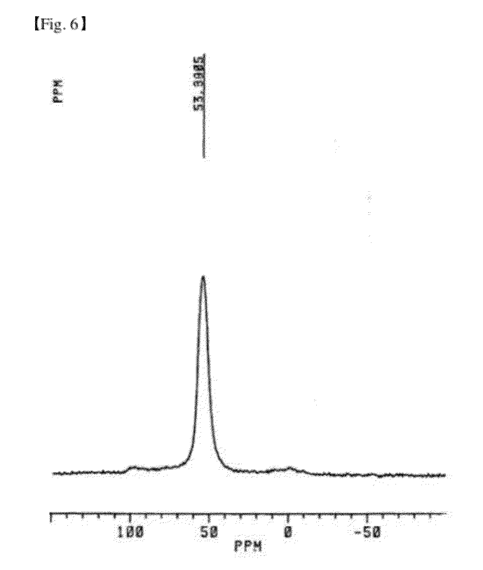

The low-angle X-ray diffraction pattern of the present substance (FIG. 4) shows that the zeolite thin film and surfactant layer are aligned regularly to form a multilamellar stacking. The high-angle X-ray diffraction (FIG. 5) was identical to the structure of a highly crystalline molecular sieve material. However, since such zeolite material has a single unit cell length to the b-crystalline axis, only the diffraction pattern corresponding to h01 diffraction is represented clearly. ²⁷Al MAS NMR spectrum of the MFI zeolite (FIG. 6) shows a peak corresponding to the chemical shift of 57-65 ppm region, which coincides with the chemical shift of a tetrahedral coordination Al represented in the crystal film zeolite structure. NMR peaks of 0-10 ppm region corresponding to Al (octahedron coordination) that exists outside the zeolite framework were not observed.

### Example 2

### Synthesis of Multilamellar Stacking MFI Aluminosilicate with Single Unit Cell Thickness by Removing an Organic Surfactant Via Calcination

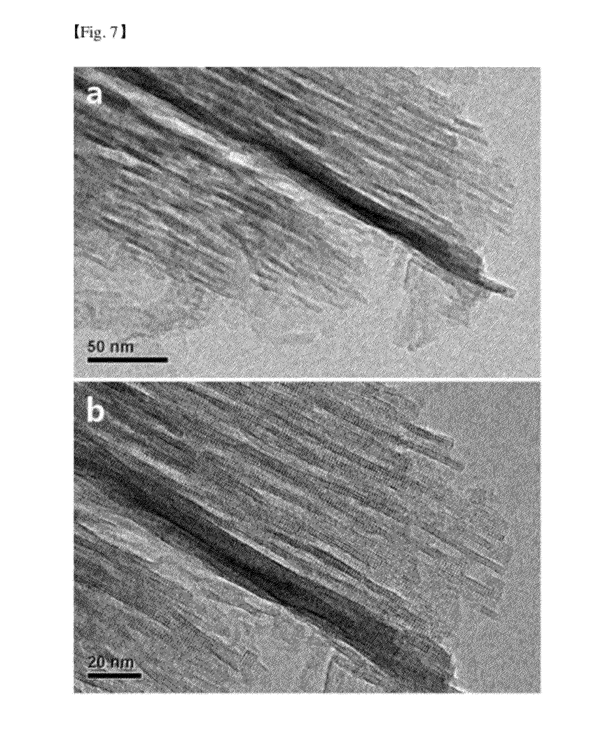

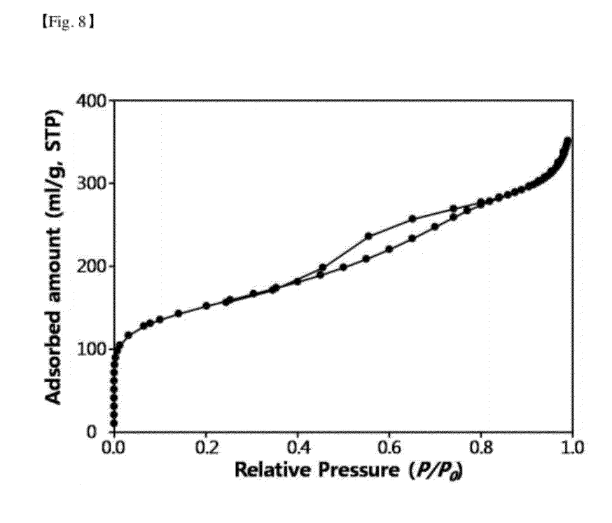

Organic surfactant layer was eliminated by calcining the multilamellar stacking MFI aluminosilicate of a single unit cell thickness synthesized in Example 1 for four hours at 550° C. As can be seen in the TEM image of FIG. 7, after the surfactant was eliminated, the zeolite thin films that were divided by a surfactant layer were condensed to an irregular structure. However, despite the condensation between irregular zeolite thin films, the zeolite framework still had a micro thickness of 2˜5 nm towards b-crystalline axis and comprised irregular mesopores between each zeolite layers. As a result of analyzing the pore structure of the product calcined through the nitrogen adsorption isotherm (FIG. 8), it was shown that the mesopore with the diameter of 2-5 nm and the pore volume of 0.7 mL/g was comprised. This zeolite material represented the BET surface area of 520 m²/g and was confirmed to have the Si/Al ratio of 43 by using ICP.

### Example 3

### Synthesis of Multilamellar MFI Aluminosilicate with a Single Unit Cell Thickness Supported by a Silica Pillar Between the Layers

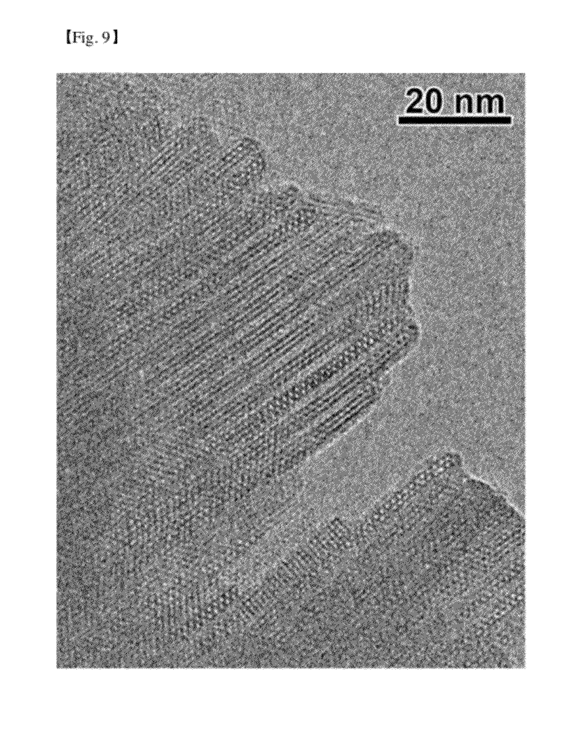

After applying 4 g of TEOS to 1 g of the multilamellar stacking MFI aluminosilicate of a single unit cell thickness prepared in Example 1, it was stirred in a sealed plastic bottle at room temperature for 24 hours. After reaction, the obtained material was filtered without being washed and dried at room temperature for 24 hours, and then after applying 20 g of a distilled water and heating it at 100° C. for 12 hours. After that, it was filtered and washed, it was obtained through filtration and was washed. After being dried at 110° C., an organic surfactant was eliminated by calcining it at 550° C. for four hours. The materials obtained after calcinations had an amorphous silica pillar between the zeolite layers. Thus, the material obtained in Example 3 had a more regular alignment between the zeolite layers than the materials obtained without a particular treatment in Example 2, and maintained the initial shape of multilamellar stacking in a perfect state (FIG. 9). The zeolite layer maintained the thickness of 2 nm like pre-calcination, and there were mesopores of 2˜3 nm between the zeolite layers. The zeolite material represented the BET surface area of 600 m²/g and was confirmed to have Si/Al ratio of 40 by using ICP.

### Example 4

### Synthesis of Multilamellar MFI Aluminosilicate with a Single Unit Cell Thickness Delaminated in Tiny Pieces

5 g of the multilamellar stacking MFI aluminosilicate of a single unit cell thickness prepared in Example 1 was dispersed in a mixed solution of 120 g of H₂O, 30 g of hexadecyltrimethylammonium bromide and 13 g of tetrapropylammonium hydroxide. After reacting this solution at 80° C. for 16 hours, it was filtered and washed with a distilled water. After drying it at 110° C., all organic materials were eliminated through calcinations for 4 hours at 550° C.

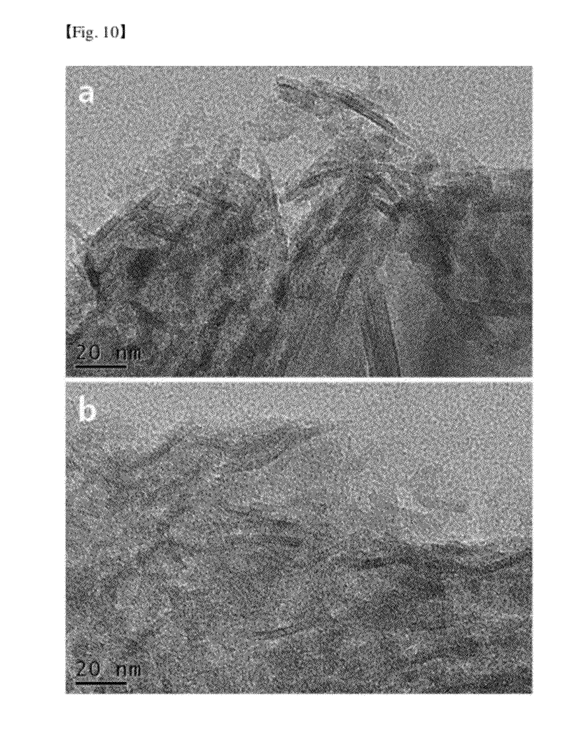

As can be seen in the TEM image of FIG. 10, the material prepared as above is a zeolite layer of a delaminated unilamellar stacking wherein the zeolite materials stacked as multilamellar structure are broken into tiny pieces and exist separately. The zeolite material represented the BET surface area of 600 m²/g and was confirmed to have Si/Al ratio of 45 by using ICP.

### Example 5

### Synthesis of Multilamellar MFI Aluminosilicate with a Single Unit Cell Thickness

It was confirmed that the synthesis of multilamellar MFI aluminosilicate of a single unit cell thickness obtained from Example 1 was possible by using 22-6-0 organic surfactants comprising one ammonium functional group and one amine functional group instead of 22-6-0 organic surfactants used in Example 1. By mixing 22-6-6 organic surfactants (organic surfactant with 22 carbon atoms of C1 and 6 carbon atoms of C2 in formula [2], comprising one ammonium functional group and one amine functional group) with TEOS, Al₂(SO₄)₃, H₂SO₄ and distilled water, a mixed gel was prepared. The mol ratio of the mixed gel is as follows:

1 Al₂O₃: 30 Na₂O: 100 SiO₂: 4000 H₂O: 18 H₂SO₄: 10 22-6-0 organic surfactant

After stirring the mixed gel at room temperature for three hours, the final mixed product was placed in a stainless autoclave and left for five days at 150° C. After cooling the autoclave to room temperature, it was filtered and washed with distilled water for several times. The obtained product was dried at 110° C.

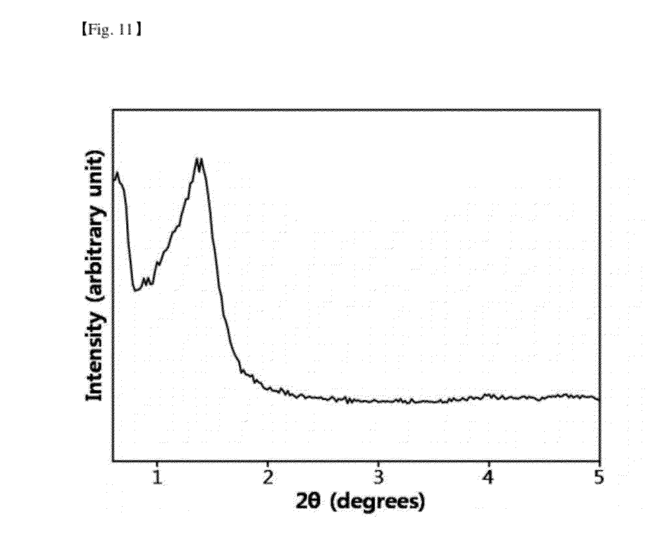

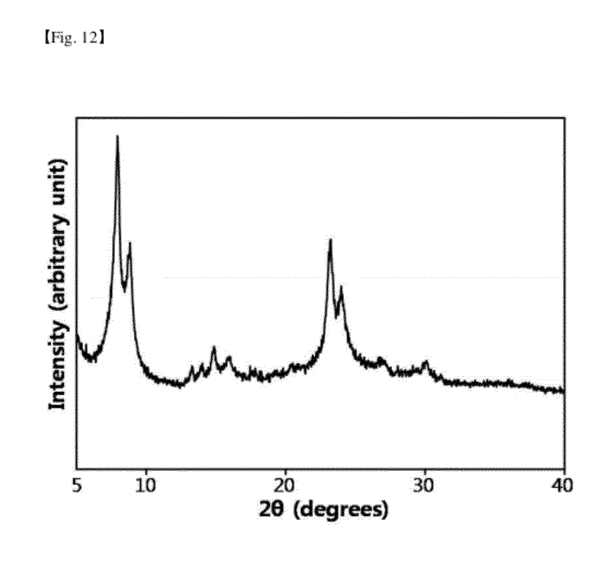

Thus, the low-angle X-ray diffraction pattern (FIG. 11) of the present material illustrates that the zeolite thin film and surfactant layers are aligned regularly to form multilamellar stacking. The high-angle X-ray diffraction (FIG. 12) shows the MFI molecular sieve having the same structure as the one having a high crystalline as obtained in Example 1.

### Example 6

### Synthesis of Multilamellar MFI Aluminosilicate with a Single Unit Cell Thickness

When aluminum was excluded in the synthesis composition of multilamellar MFI aluminosilicate with a single unit cell thickness prepared in Example 1, a multilamellar MFI silicate with a single unit cell thickness constituted only with silica could be synthesized. A mixed gel was produced by mixing 22-6-6 organic surfactants with TEOS, H₂SO₄ and distilled water. The mol ratio of the mixed gel was as follows:

30 Na₂O: 100 SiO₂: 4000 H₂O: 18 H₂SO₄: 10 22-6-6 organic surfactant

After stirring the mixed gel at room temperature for three hours, the final mixed material was placed in an autoclave and left at 150° C. for five days. After cooling the autoclave to room temperature, the product was filtered and washed with distilled water for several times. The product obtained was dried at 110° C. and then the organic material was removed therefrom though calcinations at 550° C. for four hours.

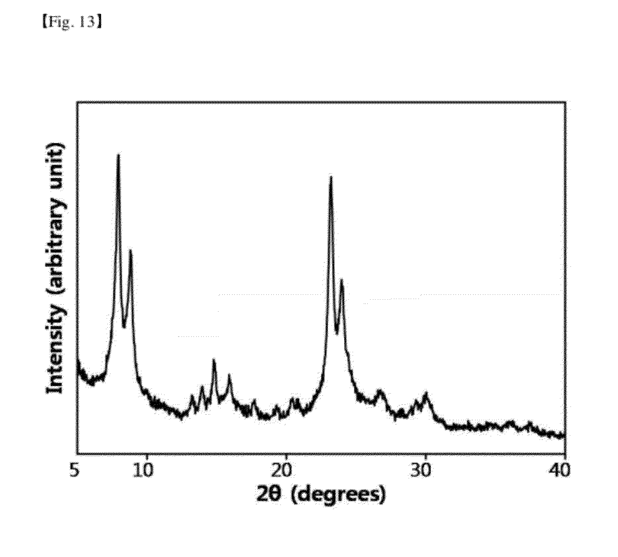

The high-angle X-ray diffraction (FIG. 13) shows it has the same structure as the MFI molecular sieve having a high crystalline as obtained in Example 1. The zeolite material represented the BET surface area of 530 m²/g and was confirmed to be constituted with pure silicate by using ICP.

### Example 7

### Synthesis of Multilamellar MFI Titanosilicalite with a Single Unit Cell Thickness

The mixed gel for synthesize of MFI titanosilicalite was prepared by mixing 22-6-6 (OH—), TEOS, titanium (IV) butoxide, and distilled water. The mol ratio of the synthesized mixed product was as follows:

0.2 TiO₂: 100 SiO₂: 4000 H₂O: 15 22-6-6 (OH—) organic surfactant

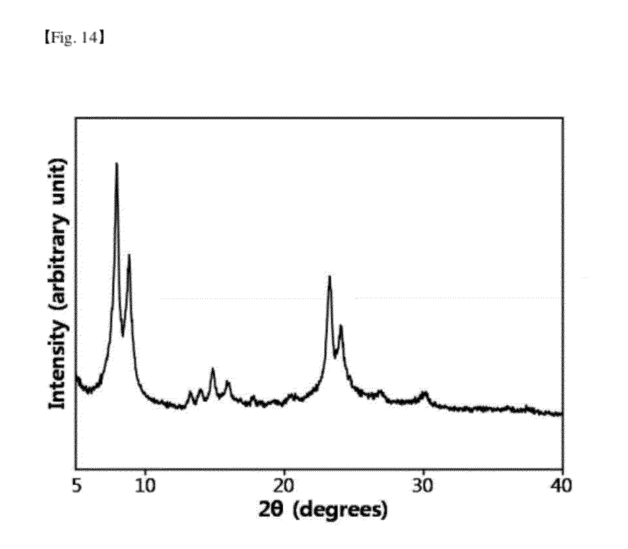

The transparent sol obtained as above was placed and sealed in a stainless autoclave, and then heated for two days at 170° C. As described above in Example 1, it was calcined after filtering the molecular sieve. The high-angle X-ray diffraction (FIG. 14) shows it has the same structure as the MFI molecular sieve having a high crystalline. The zeolite material represented the BET surface area of 535 m²/g and was confirmed to have Si/Al ratio of 42 by using ICP.

### Example 8

### Synthesis of Unilamellar MFI Aluminosilicate with a Single Unit Cell Thickness

The mixed gel was prepared by mixing 22-6-6 (OH—) organic surfactant with fumed silica, Al₂(SO₄)₃ and distilled water. The mol ratio of the synthesized gel was as follows:

1 Al₂O₃: 100 SiO₂: 6000 H₂O: 3 H₂SO₄: 15 22-6-6 (OH—) organic surfactants

After stirring the mixed gel at room temperature for three hours, the final mixed material was placed in an autoclave and left at 150° C. for five days. After cooling the autoclave to room temperature, the product was filtered and washed with distilled water for several times. The product obtained was dried at 110° C. and then the organic material was removed therefrom though calcinations at 550° C. for four hours.

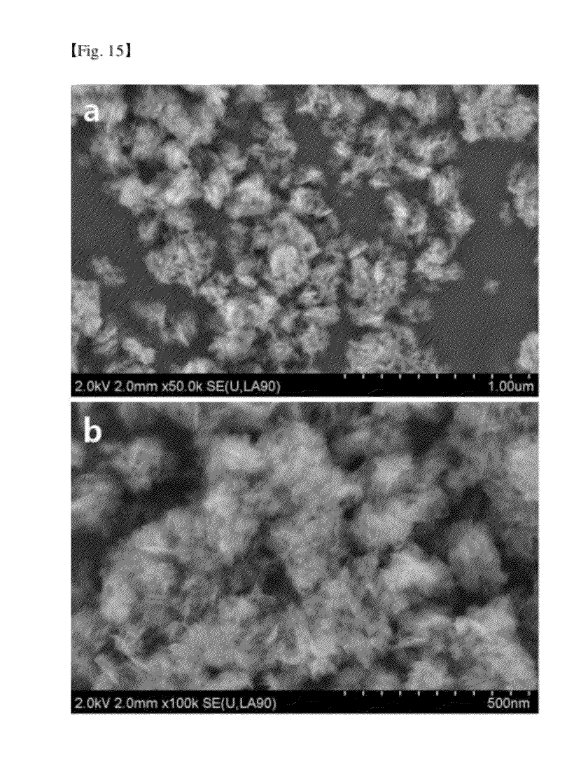

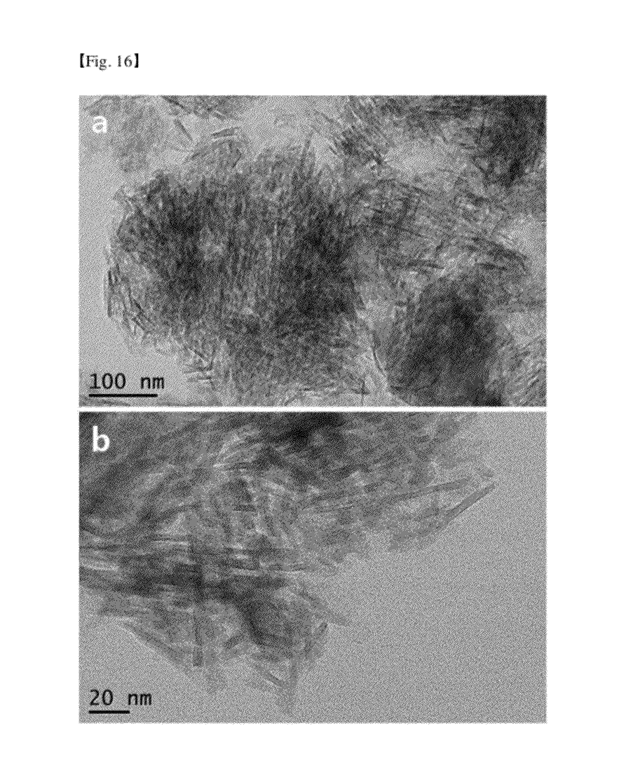

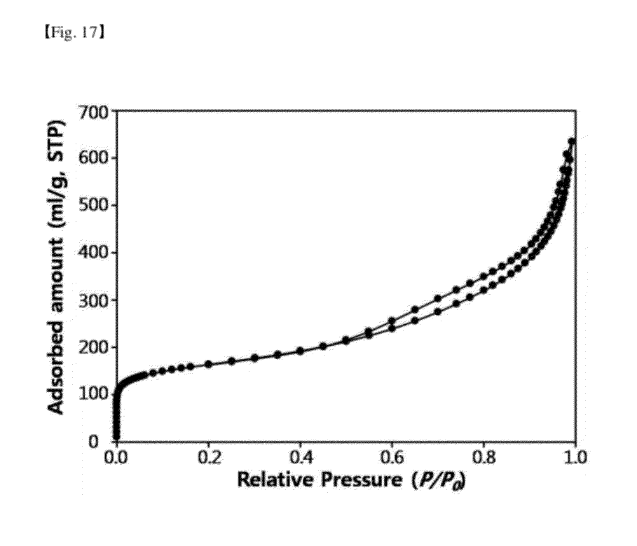

The SEM image (FIG. 15) shows that the zeolite crystal grew as a form of unilamellar structure. The TEM image (FIG. 16) shows that each unilamellar structured crystal are constituted as a MFI zeolite framework with a single unit cell thickness. Like the material obtained from Example 1, the present material has b-crystalline axis with a single unit cell size (2.0 nm) and at the same time a-axis and c-axis whose crystalline growth was restricted to below 20 nm. As a result of analyzing the pore structure of the product calcined through the nitrogen adsorption isotherm (FIG. 17), it was shown that the mesopore with the diameter of 2-10 nm and the pore volume of 0.9 mL/g was comprised. This zeolite material represented the BET surface area of 700 m²/g and was confirmed to have the Si/Al ratio of 46 by using ICP.

### Example 9

### Synthesis of Uni- or Multi-Lamellar MTW Aluminosilicate Constituted with Micro Thickness of 10 nm and Below

By adjusting the structure of the organic surfactant used in Examples 1˜8, a zeolite with a structure other than MFI or similar molecular sieve materials could be synthesized. i.e. by using 22-6-CH₂-(p-phenylene)-CH₂-6-22 organic surfactant of formula [3] below, a uni- or multi-lamellar stacking aluminosilicate constituted with nano-scale thickness of 10 nm and below could be synthesized. Here, X is a halogen (Cl, Br, I, etc.) or hydroxide group (OH), and C1, and C2 are an alkyl group which is either respectively substituted or not substituted. For synthesis, a mixed gel was prepared by mixing 22-6-CH₂-(p-phenylene)-CH₂-6-22 organic surfactants with TEOS, NaOH, Al₂(SO₄)₃, H₂SO₄ and distilled water. The mol ratio of the mixed gel was as follows:

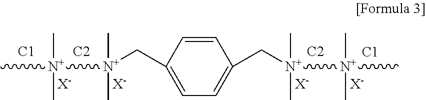

1 Al₂O₃: 23 Na₂O: 100 SiO₂: 6000 H₂O: 3 H₂SO₄: 5 22-6-CH₂-(p-phenylene)-CH₂-6-22 organic surfactant

After stirring the mixed gel at room temperature for three hours, the final mixed material was placed in an autoclave and left at 140° C. for ten days. After cooling the autoclave to room temperature, the product was filtered and washed with distilled water for several times. The product obtained was dried at 110° C.

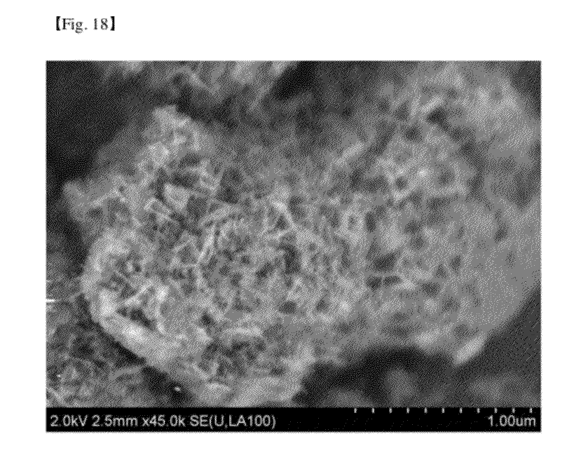

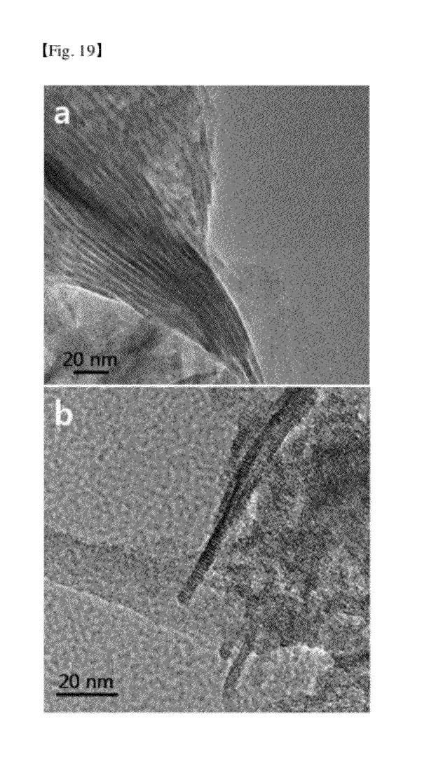

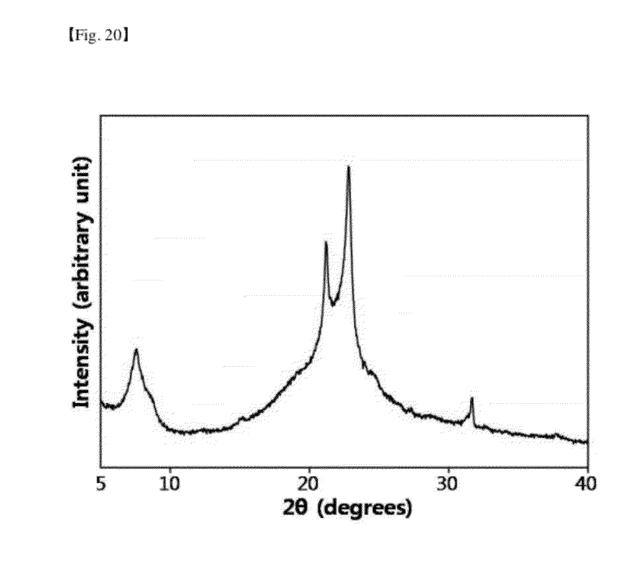

The SEM image (FIG. 18) shows that the zeolite grew as a form of lamellar structure with nano scale (20˜50 nm) thickness. FIG. 19 illustrates the TEM image of the cross section of such lamellar structured crystal, each lamellar shaped crystal is stacked on zeolite thin film with micro fine thickness of 10.0 nm and the surfactant layer of 2.0 nm, alternately and regularly to form a multilamellar stacking (FIG. 19a) or a unilamellar structure (FIG. 19b). The high-angle X-ray diffraction (FIG. 20) shows it has the same structure as the MTW molecular sieve having a high crystalline.

### Example 10

### Synthesis of Uni- or Multi-Lamellar Aluminophosphate Constituted with Micro Fine Thickness of 10 nm and Below

After mixing 22-6-6 (OH—) organic surfactant with aluminum isopropoxide and distilled water, phosphoric acid was added to prepare a mixed gel. The mol ratio of the mixed gel was as follows:

1 Al₂O₃: 1 P₂O₅: 250 H₂O: 0.5 22-6-6 (OH—) organic surfactant

After stirring the mixed gel at room temperature for three hours, the final mixed material was placed in an autoclave and left at 150° C. for four days. After cooling the autoclave to room temperature, the product was filtered and washed with distilled water for several times. The product obtained was dried at 110° C. and then the organic material was removed therefrom though calcinations at 550° C. for four hours.

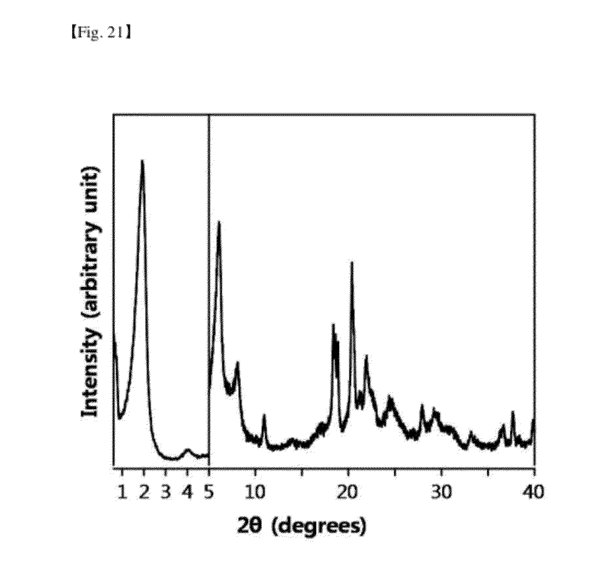

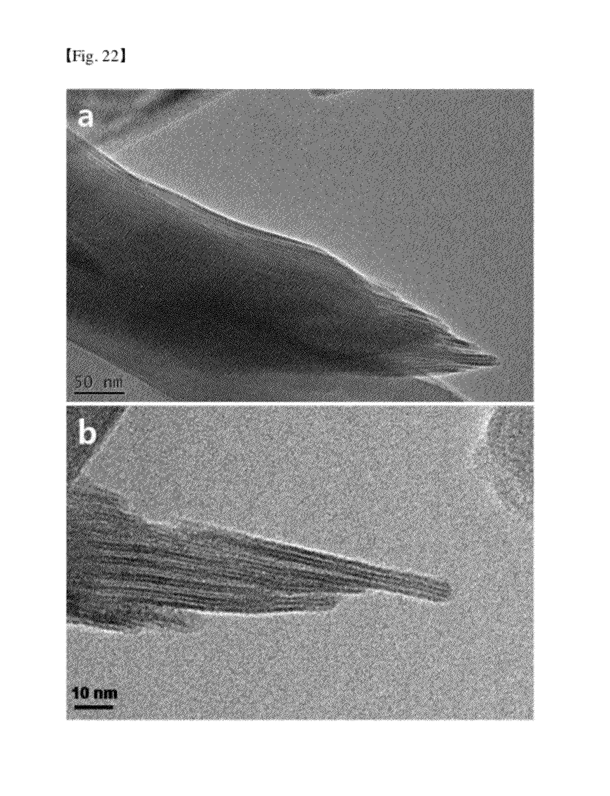

Thus, the low-angle X-ray diffraction pattern (FIG. 21, left) of the present material illustrates that the zeolite thin film and surfactant layers are aligned regularly to form multilamellar stacking. The high-angle X-ray diffraction (FIG. 21, right) shows that the present material is constituted in a framework of aluminophosphate. The TEM image (FIG. 22) shows that the framework of aluminophosphate with micro fine thickness of 2.0 nm and below and the surfactant layer are aligned alternately. It is confirmed that the Al/P ratio of the product is 1 through an ultimate analysis the MFI molecular sieve having the same structure as the one having a high crystalline as obtained in Example 1.

### Example 11

### Dealumination Reaction of Uni- or Multi-Lamellar Stacking MFI Aluminosilicate with a Single Unit Cell Thickness

2M oxalic acid of 40 mL was added to each multi- or uni lamellar MFI aluminosilicate 1 g with a single unit cell thickness prepared in Examples 2˜4, and 8, and the mixture was stirred at 65° C. for one hour under the reflux condition. After the reaction, each zeolite was filtered, washed with distilled water, and dried at 110° C., and finally calcined at 550° C. After dealumination, it is shown that the Si/Al ratio was changed from 43 to 64 in Example 2, from 40 to 60 in Example 3, from 45 to 66 in Example 4, and from 46 to 69 in Example 8 by ICP. Meanwhile, the XRD diffraction of the MFI structure was still maintained.

### Example 12

### Alkali Treatment Processing a Uni- or Multi-Lamellar MFI Aluminosilicate with a Single Unit Cell Thickness

Each multi- or uni lamellar MFI aluminosilicate 1 g with a single unit cell thickness prepared in Examples 2˜4, and 8 was applied to 0.1 M NaOH solution of 100 mL, and the dispersion solution was stirred for six hours. Then, the zeolite was filtered, washed with distilled water and dried at 110° C. The diameters of mesopore of uni- or multilamellar MFI aluminosilicates with a single unit cell thickness which were alkali-treated all increased from 2-3 nm to 4-5 nm.

### Example 13

### Exchange of Cation of Uni- or Multi-Lamellar MFI Aluminosilicate of a Single Unit Cell Thickness Using Ammonium Nitrate

Each multi- or uni-lamellar structured MFI aluminosilicate 1 g with a single unit cell thickness prepared in Examples 2˜4, and 8 was added to 0.1 M ammonium nitrate solution of 40 mL, and the solution was stirred for five hours under a reflux condition. Then, the zeolite was filtered, washed with distilled water and dried at 110° C. Finally, it was calcined at 550° C. According to the ICP analysis, it was confirmed that substantially all Na⁺ ions in the zeolite micro pores were exchanged with H⁺ ions through this process.

### Example 14

The catalytic reaction of five types included in the following example was not limited to the lamellar structure with a single unit cell thickness or multi- or uni-MFI molecular sieve materials, and the method of preparation thereof, but was carried out to show that it can be applied to various catalytic process using these materials.

A. Application of Unilamellar MFI Aluminosilicate with a Singe Unit Cell Thickness as a Reforming Catalyst of Gaseous Methanol

The unilamellar MFI aluminosilicate with a singe unit cell thickness prepared in Example 8 was exchanged with H⁺-ion through Example 13, then powder was condensed without a binding agent, and then the molecular particle of 14-20 mesh size was obtained by gridding pellet. Also, in order to compare the zeolite catalyst performance, a common MFI zeolite (ZSM-5) was prepared. The reforming reaction of methanol was performed by using a fluidized stainless reactor which was self-made (inner diameter=10 mm, outer diameter=11 mm, length=45 cm), and the reactant was analyzed by using online gas chromatography connected to the stainless reactor. The reaction process is as follows: in order to support releasing of reacting heat, a catalyst of 100 mg was mixed with 20 mesh sized sand of 500 mg was placed in a catalytic device (½″ filter GSKT-5u) of the stainless reactor; the catalyst was activated for eight hours at 550° C. under the nitrogen flow, and after cooling the reactor to 325° C. which is the reaction temperature, methanol was injected with a needle pump at the flow speed of 0.02 mL/m. Here, the velocity of the fluid of nitrogen gas was maintained at 20 mL/m, and the product was analyzed periodically by using online gaschromatography. The distribution of the product is indicated in Table 1. The unilamellar MFI aluminosilicate with a singe unit cell thickness of the present invention showed the product distribution which is remarkably different from conventional MFI catalyst.

TABLE 1

| | Unilamellar MFI zeolite | Conventional |
|---|---|---|
| | with single unit cell | MFI zeolite |
| Product distribution | thickness (%) | (%) |
| C₂H₄ | 11.4 | 42.5 |
| C₃H₆ | 51.2 | 0 |
| C₄H₈ | 8.6 | 12.6 |
| Other fatty compound | 3.3 | 13.1 |
| Benzene | 1.3 | 2.6 |
| Toluene | 1.0 | 1.4 |
| Xylene | 2.9 | 8.9 |
| Trimethylbenzene | 5.2 | 9.2 |
| C₁₀₊ | 14.6 | 9 |
| Others | 0.5 | 0.7 |
| Total | 100 | 100 |
| Selectivity for olefin (%) | 71.2 | 55.1 |
| Selectivity for gasoline (%) | 25 | 31.1 |

B. Isopropylation of Benzene

After the same material as used in Example 14A was placed in a fluidized reactor, it was activated at 550° C. After cooling the reacting temperature of the reactor to 210° C., the mixture of benzene and isopropyl alcohol (mol ratio of 6.5:1) was injected through a syringe pump at a fluid velocity of 0.005 mL/m. Here, the velocity of the fluid of nitrogen gas was maintained at 20 mL/m, and the samples were analyzed periodically by using online gaschromatography. The distribution of the product is indicated in Table 2.

TABLE 2

| | Unilamellar MFI | |
|---|---|---|
| | zeolite with single | Conventional |
| | unit cell | MFI zeolite |
| Product distribution | thickness (%) | (%) |
| C₂H₄ | 1.06 | 1.94 |
| C₃H₆ | 1.63 | 1.66 |
| C₄H₈ | 1.57 | 1.28 |
| Benzene | 86.53 | 85.6 |
| Toluene | 0 | 0 |
| Ethylbenzene | 0 | 0 |
| Cumene | 5.61 | 7.15 |
| Isobutylbenzene | 1.57 | 1.25 |
| Di-isopropylene | 0.86 | 0.37 |
| Others | 1.17 | 0.75 |
| Total | 100 | 100 |
| Selectivity for cumene (%) | 69.78 | 81.53 |
| Selectivity for di-isopropylene (%) | 10.70 | 4.22 |
| Selectivity for aromatic compound | 8.04 | 8.77 |
| (%) | | |
| Degree of conversion of benzene | 8.50 | 9.29 |
| (%) | | |

C. Liquid-Phase Condensation Reaction of Benzaldehyde and 2-Hydroxyacetophenone

The catalytic reaction was performed on the same material as used in Example 14A in a Pyrex reactor equipped with a reflux condenser. Catalyst powder of 0.1 g was activated 180° C. for two hours at, and was added to the reactor containing 2-hydroxyacetophenone of 20 mmol and benzaldehyde of 20 mmol. The reaction was carried out by stirring at 140° C. in the helium atmosphere. The reactant was analyzed periodically by using online gaschromatography. The distribution of the product is indicated in Table 3. The unilamellar MFI zeolite material with a singe unit cell thickness of the present invention showed a remarkably improved catalytic activity over conventional zeolite.

TABLE 3

| | Reaction | Degree of conver- | Product distribution (%) | |
|---|---|---|---|---|
| | time | sion of 2-hydroxy- | 2-hydroxy- | |
| Catalyst | (hr) | acetophenone (%) | chalcone | flavanone |
| Unilamellar | 5 | 18.7 | 19.6 | 80.4 |
| MFI zeolite | 24 | 50.2 | 15.6 | 84.1 |
| with single | | | | |
| unit cell | | | | |
| thickness | | | | |
| Conventional | 5 | 4.5 | 6.7 | 93.3 |
| MFI zeolite | 24 | 35.6 | 14.6 | 85.4 |

D. Synthesis of Hydrocarbon Through Reforming of Wasted Plastic

The same material as used in Example 14A was used. In the present example, solid powder of unstabilized linear low-density polyethylene was used as a standard reacting material. After placing the mixture of 10 g of polyethylene and 0.1 g of catalyst in a semi-batch Pyrex, physical stirring was performed. Here, the temperature of the reactor was increased from room temperature to 340° C. at the velocity of 6° C./m for two hours, and was maintained. A volatile product from the reaction was eliminated from the reactor by using the nitrogen flow (fluid velocity=35 mL/m), and the product was collected in a liquid and air form, respectively by using ice trap and air pocket attached to the side of the reactor. After the reaction, such liquid and air products were analyzed by using gaschromatography. The result of distribution of the product is indicated in Table 4. In the present example as well, the unilamellar MFI zeolite material with a singe unit cell thickness of the present invention showed a remarkably improved catalytic activity over conventional zeolite.

TABLE 4

| | Degree of | Selectivity (wt %) | | |
|---|---|---|---|---|
| | conversion (%) | C₁-C₅ | C₆-C₁₂ | >C₁₃ |
| Unilamellar | 81.2 | 89 | 11 | 0 |
| MFI zeolite | | | | |
| with a single | | | | |
| unit cell | | | | |
| thickness | | | | |
| Conventional | 52.1 | 95 | 5 | 0 |
| MFI-type | | | | |
| zeolite | | | | |

1. A zeolite or zeotype material comprising a regularly aligned multilamellar stacking or a randomly aligned unilamellar structure to have having a framework corresponding to a single unit cell thickness along at least one axis.

2. A zeolite or zeotype material comprising a framework comprising a multilamellar stacking or a unilamellar structure, wherein the framework comprises a connection of 10 or less single unit cells along at least one axis.

3. The zeolite or zeotype material according to claim 1, wherein the framework is a zeolite MFI framework.

4. The zeolite or zeotype material according to claim 1, wherein the framework is a zeolite MTW framework.

5. The zeolite or zeotype material according to claim 1, wherein the framework is a zeotype material AIPO (aluminophosphate) framework or other zeotype material framework.

6. The zeolite or zeotype material according to claim 1, wherein the zeolite has a chemical composition of aluminosilicate, pure silicate or titanosilicate.

7. A crystalline molecular sieve material having mesospores, the crystalline molecular sieve material prepared by calcination or chemical treatment of a zeolite or a zeotype material according to claim 1.

8. The crystalline molecular sieve material according to claim 7, wherein the crystalline molecular sieve material has a BET area of 450˜1000 m²/g, a micropore volume of 0.03˜0.15 mL/g, and a mesopore volume of 0.10˜1.0 ml/g.

9. An activated or a reformed material comprising a zeolite or a zeotype material according to claim 1, the activated or reformed material prepared using a post-treatment of the zeolite or zeotype material according to claim 1, the post-treatment selected from delamination, pillaring, basic aqueous solution treatment, ion exchange, dealumination, metal supporting or organic functionalization.

10. A method for preparing a crystalline molecular sieve material comprising:
A) forming an organic-inorganic hybrid gel comprising inorganic gel domains with nanometer size stabilized by organic gel domains by polymerizing an organic surfactant gel precursor with another gel precursor selected from silica or alumina,
B) converting the inorganic gel domains with nanometer size stabilized by organic gel domains into a zeolite by a crystallizing process, and
C) selectively eliminating the organic gel domain from the material obtained by the step B).

11. The method according to claim 10, wherein the organic surfactant is a compound selected from: 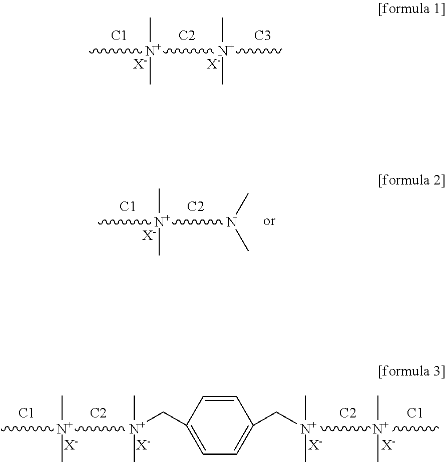
(wherein, X is a halogen (Cl, Br, I) or an hydroxide group (OH);
C1 is a substituted or an unsubstituted C₈₋₂₂ alkyl group;
C2 is a substituted or an unsubstituted C₃₋₆ alkyl group;
C3 is a substituted or an unsubstituted C₁₋₈ alkyl group or an alkenyl group, or may be various molecular structures substituted with other atoms except carbon in periodic table; and
the ammonium functional group may be extended to 2 or more and may be extended to substituted material with more various structures).

12. A zeolite or a zeotype material comprising a framework comprising a multilamellar stacking or a unilamellar structure, prepared by using an organic surfactant selected from: 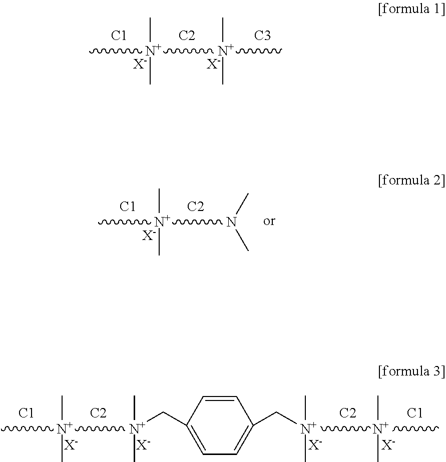
(wherein, X is a halogen (Cl, Br, I) or a hydroxide group (OH);
C1 is a substituted or an unsubstituted C₈₋₂₂ alkyl group;
C2 is a substituted or an unsubstituted C₃₋₆ alkyl group;
C3 is a substituted or an unsubstituted C₁₋₈ alkyl group or an alkenyl group, or may be various molecular structures substituted with other atoms except carbon in periodic table; and
the ammonium functional group may be extended to 2 or more and may be extended to substituted material with more various structures),
wherein the framework comprises a connection of 10 or less single unit cells along at least one axis.

13. An activated or reformed material comprising a zeolite or a zeotype material, the activated or reformed material prepared by the method of claim 10 and further comprising D) using a post-treatment of the zeolite or zeotype material according to claim 1, the post-treatment selected from delamination, pillaring, basic aqueous solution treatment, ion exchange, dealumination, metal supporting or organic functionalization.

14. The method according to claim 10, further comprising:
controlling the pore structure of the crystalline molecular sieve material by adding an additional surfactant, a polymer, an inorganic salt or an additive to the organic-inorganic hybrid gel in the step A).

15. The method according to claim 10, wherein the crystallizing process comprises hydrothermal synthesis, microwave heat or dry-gel synthesis.

16. A process comprising catalytically reforming a hydrocarbon or the substituted form thereof using a zeolite or a zeotype material according to claim 1.

17. The catalytic process according to claim 16, wherein the hydrocarbon is in a gas phase, a liquid phase, a solid phase or a mixture thereof.
